(19) United States
(12) Patent Application Publication
(10) Pub. No.: US 2005/0043649 A1
(43) Pub. Date: Feb. 24, 2005

(54) Multi-lumen manometry catheters

(76) Inventor: Robert Graham Urie, Buckinghamshire (GB)

Correspondence Address: Don W Bulson;Renner Otto Boisselle & Sklar, 19th Floor, 1621 Euclid Avenue, Cleveland, OH, 44115, US

(21) Appl. No.: 10/488,800

(22) PCT Filed: Aug. 30, 2002
(86) PCT No.: PCT/GB02/03980

(30) Foreign Application Priority Data
Sep. 6, 2001 (GB) 01215441

(51) Int. Cl. A61M 5/00; A61M 25/00; A61B 5/00

(52) U.S. Cl. 600/561; 600/593; 600/587; 604/264

(57) ABSTRACT

A manometry apparatus includes a multi-lumen catheter comprising an extruded flexible tube having a plurality of lumens extending through the body of the tube between a proximal end and a distal end. The flexible tube has a first diameter in a proximal portion extending over a part of its length at the proximal end and a second diameter, larger than the first diameter, in a distal portion extending over a part of its length at the distal end. A plurality of flexible connection tubes are each coupled to a respective one of the plurality of lumens by insertion of the respective flexible connection tube into a corresponding lumen. Connection of the multi-lumen catheter to multiple flexible connection tubes is simplified by extruding the distal end of the catheter to a larger dimension so that the connection tubes can be directly inserted into the catheter lumens.

The present invention relates to multi-lumen tubes for use as manometry catheters, and in particular to improvements in the connection mechanisms for such catheters.

Various medical diagnosis procedures require the use of manometers that are capable of measuring pressure simultaneously at multiple locations along the length of a tube. For example, in gastrointestinal manometry, a manometry catheter consists of a multi-lumen tube in which each individual lumen communicates with a lumen opening, with the multiple lumen openings being located at various points along the length of the tube.

The multi-lumen tube, with its plurality of lumen openings, is positioned within the tube cavity in which pressure is to be measured, for example, the oesophagus. The lumen openings enable sensing of the pressure in various parts of the oesophagus along its length. As peristaltic waves from the oesophagus apply pressure to varying positions along the tube, these pressures are communicated to pressure transducers coupled to a distal end of each lumen.

To obtain comprehensive and useful pressure readings from many positions along the oesophagus, a corresponding number of lumens must be used within the multi-lumen tube. In a typical example, a nine-lumen tube is used.

A problem encountered is that the multi-lumen tube to be inserted into the patient's body must of necessity be sufficiently small in diameter so that patient discomfort is minimised, so that undue distortion of the oesophagus is avoided and so that adverse effects on the pressure measurement are also minimised.

Although small diameter multi-lumen tubes can readily be formed from suitable flexible plastics materials, easy and reliable connection of each of the many lumens, at a distal end of the catheter, to separate pressure transducers can be a problem owing to the very small internal diameter of the individual lumens.

The present invention aims to provide an improved means for coupling the individual lumens of a multi-lumen catheter tube at a distal end thereof to individual pressure transducer couplings.

According to one aspect, the present invention provides a manometry catheter comprising an extruded flexible tube haying a plurality of lumens extending through the body of the tube between a proximal end and a distal end, the flexible tube having a first diameter in a proximal portion extending over a part of its length at the proximal end and a second diameter, larger than the first diameter, in a distal portion extending over a part of its length at the distal end.

According to another aspect, the present invention provides a manometry apparatus comprising:
a multi-lumen catheter comprising an extruded flexible tube having a plurality of lumens extending through the body of the tube between a proximal end and a distal end, the flexible tube having a first diameter in a proximal portion extending over a part of its length at the proximal end and a second diameter, larger than the first diameter, in a distal portion extending over a part of its length at the distal end; and
a plurality of flexible connection tubes each being coupled to a respective one of the plurality of lumens by insertion of the respective flexible connection tube into a corresponding lumen.

Embodiments of the present invention will now be described by way of example and with reference to the accompanying drawings in which:

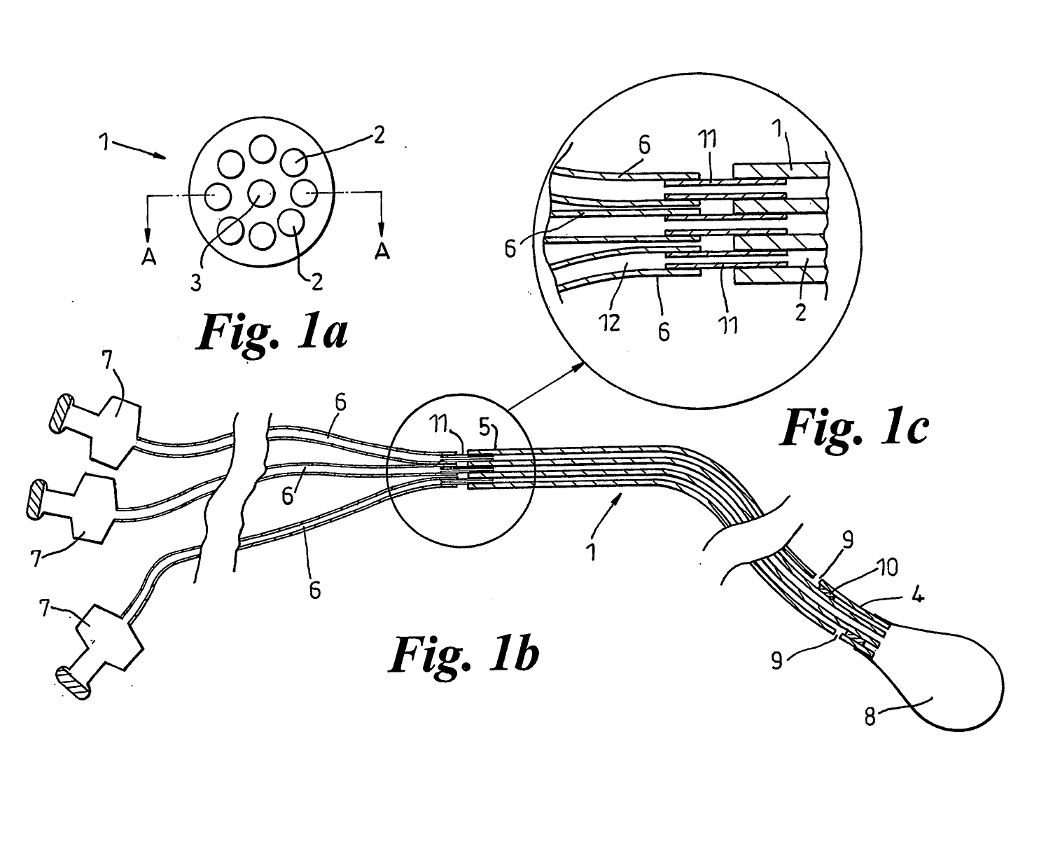

FIG. 1a is a cross-sectional end view of a nine-lumen manometry catheter of the prior art;

FIG. 1b is a schematic side cross-sectional view of a manometry catheter apparatus including connecting device according to the prior art, with the catheter shown in cross section on A-A;

FIG. 1c is a schematic close up side cross-sectional view of the connecting device of FIG. 1b;

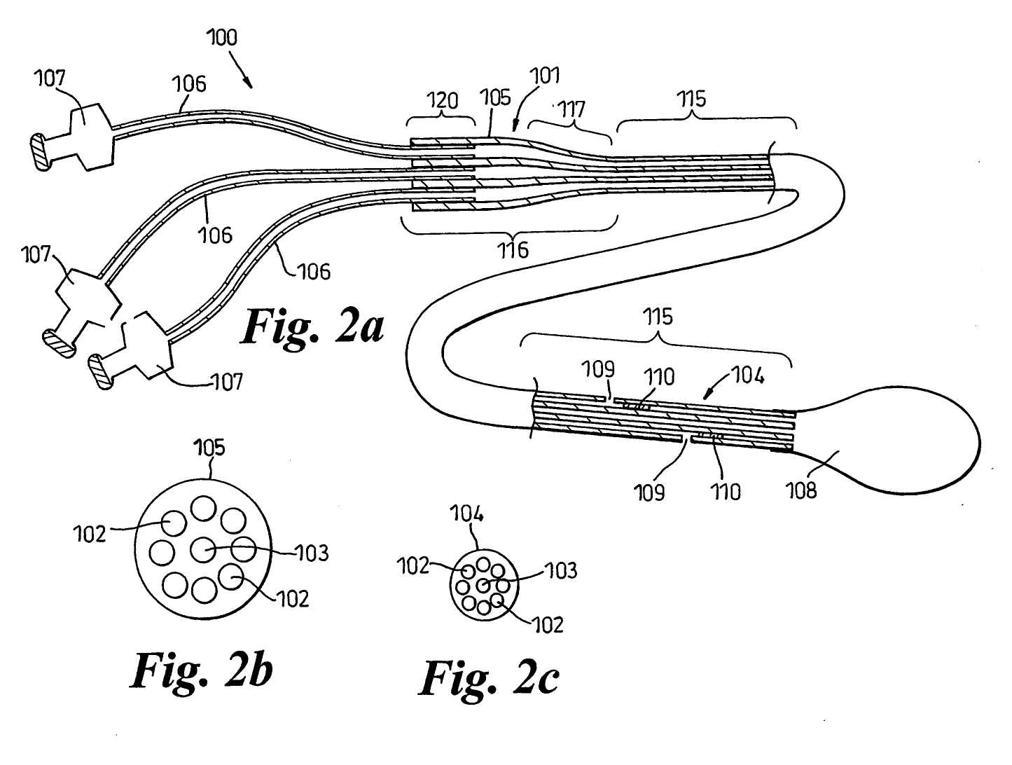

FIG. 2a is a schematic side view of a manometry catheter apparatus according to one embodiment of the present invention;

FIG. 2b is a cross-sectional end view of the distal end of the catheter in FIG. 2a; and

FIG. 2c is a cross-sectional end view of the proximal end of the catheter of FIG. 2a.

With reference to FIG. 1a, a conventional multi-lumen tube catheter 1 has a diameter of approximately of 5 mm. The catheter has eight peripheral lumens 2 and one central lumen 3 each having an internal diameter of approximately 1 mm. The catheter is conventionally extruded in a flexible material such as PVC, using known extrusion processes.

With reference to FIG. 1b, a conventional manometry apparatus includes a multi-lumen catheter 1 extending from a proximal end 4 to a distal end 5. The apparatus further includes a plurality of flexible connection tubes 6 each connected at a first end to the distal end 5 of the multi-lumen catheter 1 and coupled at the second end to a plug 7. Each plug 7 is adapted to be received by, and form an airtight seal with, a pressure sensing device.

At the proximal end 4 of the catheter 1 an inflation device, such as balloon 8 is coupled to the multi-lumen catheter 1 by way of a friction fit and/or adhesive or other suitable means. The balloon may be inflated by way of the central lumen 3, coupled to one of the flexible connection tubes 6 and corresponding plug 7.

To fulfil the objective of simultaneous pressure measurement at multiple positions along the length of the catheter 1, each peripheral lumen 2 communicates with a lumen opening 9 comprising an aperture in the outer wall of the multi-lumen tube 1. Adjacent to the lumen opening 9, and between the lumen opening and the proximal end of the catheter 1 the remaining portion of the lumen 2 is occluded by an occlusion 10 to prevent communication with the proximal end of the catheter 1. The occlusion 10 may be any suitable device or substance adequate to form an airtight seal within the lumen, such as a pin inserted into the proximal end 4 and glued into place.

With reference to FIG. 1c, to couple the catheter 1 to the flexible connection tubes 6 a plurality of hollow cylindrical pins 11 are engaged in each lumen 2, 3 of the multi-lumen catheter 1, and correspondingly into the lumen 12 of a respective flexible connection tube 6. Typically, the hollow cylindrical pins 11 are formed of stainless steel and are glued into place.

It will be noted that the connection of nine stainless steel pins 11 in between the multi-lumen catheter 1 and the plurality of flexible connection tubes 6 incurs manufacturing costs and assembly costs in producing such fine, high quality metal components and installing them accordingly.

It will also be noted that the hollow cylindrical pins 11 also impose a reduced internal diameter on the lumen communication between the catheter 1 and the flexible connection tubes 6. Of necessity, the internal diameter of the hollow cylindrical pins 11 must be less than that of either the catheter lumens 2, 3 or the lumen 12 of the flexible connection tubes 6. This is disadvantageous for a number of reasons.

Firstly, it effectively imposes a lower limit on the internal diameter of the lumens being connected, according to the availability of small diameter hollow cylindrical pins. This limits the number of lumens that can be provided within a catheter of an external diameter dictated by the medical conditions in which it is to be used.

Secondly, the reduced internal diameter of the hollow cylindrical pins has adverse effects on the fluid flow through the combined lumen/connector system. If insufficient fluid flow is achieved through the limited internal diameter of the hollow cylindrical pins, this has an adverse effect on the accuracy and resolution of pressure measurements taken therethrough. Similarly, if the internal diameter of the hollow cylindrical pins becomes too small, capillary action can start to have adverse effects on the fluid flows upon which pressure measurements are based, resulting in inaccuracies in the measurements.

It will be shown that the present invention avoids the use of the hollow cylindrical pins 11 to connect the multi-lumen catheter 1 to the flexible connection tubes 6.

With reference to FIG. 2a, a manometry apparatus 100 according to a presently preferred embodiment of the invention includes a multi-lumen catheter 101 which has a proximal end 104 and a distal end 105 respectively seen in cross-section in FIGS. 2c and 2b. The proximal end 104 comprises a proximal portion 115 that has a first diameter. The distal end 105 comprises a distal portion 116 that has a second diameter that is larger than the first diameter.

Preferably, the proximal portion 115 extends over most of the length of the catheter, eg. all excepting about 2 to 7.5 cm, or more preferably, about 2 to 5 cm at the distal end 105 Preferably, the distal portion 116 extends over a short length of the catheter, eg. between 1 or 2 and 10 cm, or more preferably between 1 and 5 cm, or between 2 and 5 cm. Preferably, the external diameter of the proximal portion 115 is approximately 5 mm and the external diameter of the distal portion 116 is approximately 8 mm.

In the preferred embodiment, the catheter 101 has eight peripheral lumens 102 and one central lumen 103. Preferably, the peripheral lumens 102 each have an internal diameter in the proximal portion 115 of between 0.4 mm and 1 mm, and an internal diameter in the distal portion of approximately 2 mm. In one arrangement, the central lumen 103 is conveniently slightly larger, eg. between 0.7 mm and 1.5 mm internal diameter in the proximal portion 104 and approximately 2.5 mm internal diameter in the distal portion 105.

Currently preferred sizes (diameters) of nine-lumen catheter required for various medical applications are as shown in the table below:

| | | | LUMEN INTERNAL [symbol] | | | |
|---|---|---|---|---|---|---|
| | OUTSIDE [symbol] | | PROXIMAL END | | DISTAL END | |
| | PROXIMAL | DISTAL | | PERIPH- | | PERIPH- |
| TYPE | END | END | CENTRAL | ERAL | CENTRAL | ERAL |
| A | 4.9 mm | 7.9 mm | 1.5 mm | 0.85 mm | 2.5 mm | 2.0 mm |
| B | 3.9 mm | 7.9 mm | 1.2 mm | 0.6 mm | 2.5 mm | 2.0 mm |
| C | 2.3 mm | 7.9 mm | 0.74 mm | 0.46 mm | 2.5 mm | 2.0 mm |

The catheter is preferably extruded in a flexible material such as PVC, polyurethane or any suitable medical grade thermoplastic polymer according to known extrusion processes.

In the present invention, it has been recognised that conventional extrusion process techniques can cheaply and easily be deployed to increase the diameter of the catheter 101 by slowing up the extrusion process for the final few centimetres of the catheter, resulting in a tapering outward, then final larger, diameter of catheter. It is also possible, during the extrusion process, to apply an increased air pressure in the lumens of the tube being extruded, to control the increase in diameter of the catheter and its lumens.

In general, both the lateral separation of the individual lumens and the internal diameter of the individual lumens increase between the proximal portion 115 and the distal portion 116. Preferably, this increase occurs in a taper zone 117 resulting from a gradual change in the extrusion process.

The manometry apparatus 100 includes the multi-lumen catheter 101 together with a plurality of flexible connection tubes 106 each connected at a first end to the distal end 105 of the multi-lumen catheter 101 and coupled at the second end to a plug 107. Each plug 107 is adapted to be received by, and form an airtight seal with, a pressure sensing device (not shown).

For certain medical applications, such as use of catheters for ano-rectal measurements, an inflation device, such as balloon 108 may be coupled to the proximal end 104 of the multi-lumen catheter 101 by way of a friction fit and/or adhesive or other suitable means. The balloon may be inflated by way of the central lumen 103, coupled to one of the flexible connection tubes 106 and corresponding plug 107.

Each peripheral lumen 102 communicates with a lumen opening 109 comprising an aperture in the outer wall of the multi-lumen tube 101. Adjacent to each lumen opening 109, and between the lumen opening and the proximal end of the catheter 101, the remaining portion of the lumen 102 is occluded by an occlusion 110 to prevent communication with the proximal end of the catheter. The occlusion 110 may be any suitable device or substance adequate to form an airtight seal within the lumen, such as a pin inserted into the proximal end 104 and glued into place. Alternatively, the occlusion 110 may be formed by injected glue or resin.

The invention provides for simpler and more efficient coupling of the multi-lumen catheter 101 to the flexible connection tubes 106. Because the distal portion 116 of the multi-lumen catheter is expanded during the extrusion process, direct coupling of each of the flexible connection tubes 106 into a corresponding lumen 102, 103 of the multi-lumen catheter 101 is possible. A simple friction fit between the overlapping lumen 102, 103 walls and tube 106 walls in a connection zone 120 may be used, or more preferably, an adhesive may be used.

It will be noted that the direct insertion of flexible connection tubes 106 into the lumens of the multi-lumen catheter 101 ensures that the smallest lumen diameter encountered between the lumen openings 109 and the pressure sensor plugs 107 is the internal diameter of the flexible connection tubes 106. There is no intermediate restriction of any connecting pins 11.

It will also be noted that the manufacturing costs of fine, high quality metal components 11 and the associated assembly costs for installing them are accordingly avoided.

Although the invention has been described and illustrated in the context of a nine-lumen manometry apparatus, it will be understood that the principle applies to other numbers of lumens in a multi-lumen catheter.

Other embodiments are within the scope of the appended claims.

1. (original) a manometry catheter comprising an extruded flexible tube having a plurality of lumens extending through the body of the tube between a proximal end and a distal end, the flexible tube having a first diameter in a proximal portion extending over a part of its length at the proximal end and a second diameter, larger than the first diameter, in a distal portion extending over a part of its length at the distal end.

2. The manometry catheter of claim 1 in which the proximal portion extends for a substantial part of the length of the catheter and the distal portion extends for a relatively small part of the length of the catheter.

3. The manometry catheter of claim 2 in which the distal portion has a length in the range 1 to 10 cm.

4. The manometry catheter of claim 2 in which the distal portion has a length in the range 1 to 5 cm.

5. The manometry catheter of claim 1 in which the lateral separation of the individual lumens within the flexible tube is increased within the distal portion.

6. The manometry catheter of claim 1 in which the internal diameter of the individual lumens within the flexible tube is increased within the distal portion.

7. The manometry catheter of claim 1 in which the outside diameter of the proximal portion lies in the range approximately 2 mm to 5 mm and the outside diameter of the distal portion is approximately 8 mm.

8. The manometry catheter of claim 1 in which the inside diameter of lumens located adjacent the periphery of the catheter lies in the range approximately 0.74 mm to 1.5 mm in the proximal portion and approximately 2 mm in the distal portion.

9. The manometry catheter claim 1 further including a lumen opening communicating with each of a plurality of said lumens extending through the catheter walls to an outer surface thereof.

10. The manometry catheter of claim 9 in which each lumen opening is located at a different longitudinal position along the catheter.

11. The manometry catheter of claim 1 in which the distal portion includes a tapered region between the distal end and the proximal portion.

12. A manometry apparatus comprising:
a multi-lumen catheter comprising an extruded flexible tube having a plurality of lumens extending through the body of the tube between a proximal end and a distal end, the flexible tube having a first diameter in a proximal portion extending over a part of its length at the proximal end and a second diameter, larger than the first diameter, in a distal portion extending over a part of its length at the distal end;
a plurality of flexible connection tubes each being coupled to a respective one of the plurality of lumens by insertion of the respective flexible connection tube into a corresponding lumen.
